(19) United States
(12) Patent Application Publication
(10) Pub. No.: US 2024/0123430 A1
(43) Pub. Date: Apr. 18, 2024

(54) CATALYST MATERIALS WITH TUNABLE ACTIVITY

(71) Applicant: NOVA CHEMICALS (INTERNATIONAL) S.A., Fribourg (CH)

(72) Inventors: Vasily SIMANZHENKOV, Calgary (CA); Xiaoliang GAO, Calgary (CA); Marie BARNES, Calgary (CA); Perry DE WIT, Vernon (CA)

(21) Appl. No.: 18/278,972

(22) PCT Filed: Feb. 17, 2022
(86) PCT No.: PCT/IB2022/051431

§ 371 Date: Aug. 25, 2023

Related U.S. Application Data
(60) Provisional application No. 63/154,457, filed on Feb. 26, 2021.

(51) Int. Cl. B01J 23/00; B01J 27/057; B01J 35/40; B01J 35/61; B01J 35/63; B01J 35/70; B01J 37/08; C07C 5/32

(52) U.S. Cl. CPC B01J 23/002; B01J 27/0576; B01J 35/40; B01J 35/612; B01J 35/633; B01J 35/70; B01J 37/08; C07C 5/324; B01J 2523/21; B01J 2523/55; B01J 2523/56; B01J 2523/64; B01J 2523/68; C07C 2523/20; C07C 2523/22; C07C 2523/24; C07C 2523/32

(57) ABSTRACT

A catalyst material includes molybdenum (Mo): vanadium (V). the molar ratio of Mo:V being between 1:0.12 and 1:0.49; tellurium (Te), the molar ratio of Mo:Te being between 1:0.01 and 1:0.30; niobium (Nb), the molar ratio of Mo:Nb being between 1:0.01 and 1:0.30; and beryllium (Be), the molar ratio of Mo:Be being from 1:1 to 1:50.

### CLAIM OF PRIORITY

This application claims priority to U.S. Provisional Application No. 62/110,457 filed on Feb. 26, 2021, the entire contents of which are hereby incorporated by reference.

### BACKGROUND ART

Selective oxidation processes, such as oxidative dehydrogenation (ODH), are an alternative to steam cracking that are exothermic and produce little or no coke. In ODH, a lower alkane, such as ethane, is mixed with oxygen in the presence of a catalyst and optionally an inert diluent, such as carbon dioxide or nitrogen or steam, which may be performed at temperatures as low as 300° C., to produce the corresponding alkene. Various other oxidation products may be produced in this process, including carbon dioxide and acetic acid, among others. ODH suffers from lower conversion rates when compared to steam cracking, a fact that when combined with lower selectivity and the risk of deflagration, explosion, or thermal reaction due to mixing of a hydrocarbon with oxygen, may have prevented ODH from achieving widespread commercial implementation.

Successful commercial implementation of ODH requires a catalyst with sufficient activity and selectivity to the desired product but may be supported by recovering energy from the heat produced, for example in the production of high-pressure steam. Mixed metal oxide catalysts well known for use in ethane ODH are typically suited for operating at temperatures below 400° C. in order to maintain acceptable selectivity to ethylene. At these temperatures production of high-pressure steam is inefficient. Catalysts that work at higher temperatures, for example above 400° C., are generally associated with higher conversions but with a lower selectivity to ethylene. Development of a catalyst or means for operating at higher temperatures where energy recovery is more efficient, and conversion and selectivity are sufficient should prove to be valuable in the commercialization of ethane ODH.

### SUMMARY OF INVENTION

In an aspect, a catalyst material includes molybdenum (Mo); vanadium (V), the molar ratio of Mo:V being between 1:0.12 and 1:0.49; tellurium (Te), the molar ratio of Mo:Te being between 1:0.01 and 1:0.30; niobium (Nb), the molar ratio of Mo:Nb being between 1:0.01 and 1:0.30; and beryllium (Be), the molar ratio of Mo:Be being less than 1:1.

Embodiments can include any combination of one or more of the following features.

The molar ratio of Mo:Be is between 1:1 and 1:8. An activity of the catalyst material is higher than an activity of a catalyst corresponding to the catalyst material. The catalyst corresponding to the catalyst material comprises a mixed metal oxide comprising Mo_{1.0}V_{0.12-0.49}Te_{0.01-0.30}Nb_{0.01-0.30}O_{d}. A 35% conversion temperature of the catalyst material is between 370° C. and 390° C.

The molar ratio of Mo:Be is less than 1:8. The molar ratio of Mo:Be is between 1:8 and 1:50. An activity of the catalyst material is lower than an activity of a catalyst corresponding to the catalyst material. A 35% conversion temperature of the catalyst material is between 400° C. and 410° C.

A selectivity of the catalyst material to ethylene is between 90% and 100, e.g., between 95% and 100%. The selectivity of the catalyst material to ethylene is between 90% and 100% at a temperature of between 400° C. and 500° C.

The catalyst material comprises a mixed metal oxide comprising Mo_{1.0}V_{0.12-0.49}Te_{0.01-0.30}Nb_{0.01-0.30}O_{d}, in which d is a number to satisfy a valence of the mixed metal oxide. The molar ratio of Mo:V is between 1:0.20 and 1:0.45; the molar ratio of Mo:Te is between 1:0.05 and 1:0.25; and the molar ratio of Mo:Nb is between 1:0.05 and 1:0.25. The molar ratio of Mo:V is between 1:0.25 and 1:0.40; the molar ratio of Mo:Te is between 1:0.07 and 1:0.20; and the molar ratio of Mo:Nb is between 1:0.10 and 1:0.20. The molar ratio of Mo:V is between 1:0.30 and 1:0.35; the molar ratio of Mo:Te is between 1:0.10 and 1:0.17; and the molar ratio of Mo:Nb is between 1:0.12 and 1:0.15.

Molar ratios of Mo:V. Mo:Te, and Mo:Nb are determined by elemental analysis using inductively coupled plasma mass spectrometry (ICP-MS).

In an aspect, a method of making a catalyst material includes forming an aqueous mixture comprising a catalyst comprising a mixed metal oxide comprising Mo_{1.0}V_{0.12-0.49}Te_{0.01-0.30}Nb_{0.01-0.30}O_{d}, in which d is a number to satisfy a valence of the mixed metal oxide; and an additive comprising Be. The method includes heating the aqueous mixture to form a paste; and baking the paste to form the catalyst material.

Embodiments can include any combination of one or more of the following features.

Forming an aqueous mixture comprises forming an aqueous mixture in which a weight ratio of the catalyst to the additive is less than 92:8, e.g., less than 80:20.

Forming an aqueous mixture comprises forming an aqueous mixture comprising the catalyst and BeO.

The method includes drying the paste.

Baking the paste comprises calcining the paste.

The method includes calcining the paste at a temperature of between 330° C. and 380° C., e.g., at a temperature of 350° C.

The catalyst material includes molybdenum (Mo); vanadium (V), the molar ratio of Mo:V being between 1:0.12 and 1:0.49; tellurium (Te), the molar ratio of Mo:Te being between 1:0.01 and 1:0.30; niobium (Nb), the molar ratio of Mo:Nb being between 1:0.01 and 1:0.30; and beryllium (Be), the molar ratio of Mo:Be being less than 1:1.

In an aspect, a method for generation of ethylene from ethane includes processing ethane in a reactor in an oxidative dehydrogenation process in the presence of a catalyst material. The catalyst material includes molybdenum (Mo); vanadium (V), the molar ratio of Mo:V being between 1:0.12 and 1:0.49; tellurium (Te), the molar ratio of Mo:Te being between 1:0.01 and 1:0.30; niobium (Nb), the molar ratio of Mo:Nb being between 1:0.01 and 1:0.30; and beryllium (Be), the molar ratio of Mo:Be being less than 1:1.

Embodiments can include comprising processing the ethane at a temperature between 400° C. and 500° C.

In an aspect, a method includes tuning an activity of a catalyst by incorporating beryllium (Be) into the catalyst, comprising adding a first quantity of Be to the catalyst to form a first catalyst material, in which an activity of the first catalyst material is higher than an activity of the catalyst; and adding a second quantity of Be to the catalyst to form a second catalyst material, in which an activity of the second catalyst material is lower than the activity of the catalyst. A selectivity of the first catalyst material is equal to the selectivity of the second catalyst material.

Embodiments can include one or more of the following features.

The catalyst comprises molybdenum (Mo). Adding a first quantity of Be comprises adding Be in a molar ratio of Mo:Be of between 1:1 and 1:8. Adding a second quantity of Be comprises adding Be in a molar ratio of Mo:Be of less than 1:8.

The approaches described here can have one or more of the following advantages. The activity of the catalyst materials described here can be tuned by controlling the molar ratio of molybdenum to beryllium in the catalyst materials, while maintaining a high selectivity of the catalyst materials to ethylene. The ability to tune the activity of the catalyst materials enables catalyst materials to be tailored for high selectivity at high operating temperatures, rendering the catalyst materials useful under a variety of reaction conditions. Catalyst material catalyst material with lower activity and high selectivity to ethylene also can be useful, e.g., for catalyzing reactions while reducing the production of high pressure steam or while activating different cooling media.

The details of one or more implementations are set forth in the accompanying drawings and the description below. Other features and advantages will be apparent from the description and drawings, and from the claims.

### BRIEF DESCRIPTION OF DRAWINGS

FIG. 1 shows a cross section schematic of a microreactor unit (MRU) setup.

FIG. 2 shows a plot of pore volume (cm³/g) versus pore area (m²/g) for catalyst 1.2

FIG. 3 shows a plot of pore volume (cm³/g) versus pore area (m²/g) for catalyst materials 2.1. 2.2. 2.3, and 2.5

FIG. 4 shows a plot of percent pore are (%) versus pore width (Å) for catalyst 1.2.

FIG. 5 shows a plot of percent pore are (%) versus pore width (Å) for catalyst materials 2.1. 2.2, 2.3, and 2.5.

FIG. 6 shows scanning electron microscopy images for (A) catalyst material 2.5 at 1,000× magnification, (B) catalyst material 2.5 at 20,000× magnification, (C) catalyst material 2.3 at 1,000× magnification, and (D) catalyst material at 20,000× magnification.

FIG. 7 shows the X-ray diffraction (XRD) spectra for catalyst 1.2, catalyst materials 2.1-2.3 and 2.5, and beryllium oxide.

### DESCRIPTION OF EMBODIMENTS

We describe here catalyst materials, used for the oxidative dehydrogenation of alkanes, that have an activity that can be tuned depending on the ratio of beryllium to molybdenum in the catalyst materials. The catalyst materials are formed by combining a mixed metal oxide catalyst, such as a catalyst containing molybdenum (Mo), with an additive including beryllium (Be). When the molar ratio of Mo:Be in the catalyst material is lower than a threshold, such as less than 1:8, the activity of the catalyst material is decreased relative to the activity of the catalyst. When the molar ratio of Mo:Be in the catalyst material is higher than the threshold, the activity of the catalyst material is increased. The selectivity of the catalyst materials to ethylene is high, e.g., between 95% and 100%, regardless of the amount of Be in the catalyst materials.

A catalyst material refers to a material that can promote the oxidative dehydrogenation of ethane to ethylene. The catalyst material can be a plurality of particles or a formed catalyst material. Non-limiting examples of formed catalyst materials include extruded catalyst materials, pressed catalyst materials, and cast catalyst materials. Non-limiting examples of pressed and cast catalyst materials includes pellets—such as tablets, ovals, and spherical particles.

### Composition

The catalyst materials described here include molybdenum, vanadium, tellurium, niobium, beryllium, oxygen, and, optionally, sulfur. The molar ratio of molybdenum to vanadium is from 1:0.12 to 1:0.49. The molar ratio of molybdenum to tellurium is from 1:0.01 to 1:0.30. The molar ratio of molybdenum to niobium is from 1:0.01 to 1:0.30. The molar ratio of molybdenum to beryllium is less than 1:1, e.g., between 1:1 and 1:8, or less than 1:8, such as from 1:8 to 1:50. Oxygen is present at least in an amount to satisfy the valency of any present metal oxides. The molar ratios of Mo:V, Mo:Te, Mo:Nb, and Mo:Be are determined by elemental analysis using ICP-MS. In some embodiments, sulfur, when present, comprises less than 0.01 wt. % of the catalyst material.

In some embodiments, the molar ratio of molybdenum to vanadium is from 1:0.20 to 1:0.45, the molar ratio of molybdenum to tellurium is from 1:0.05 to 1:0.25, the molar ratio of molybdenum to niobium is from 1:0.05 to 1:0.25, and the molar ratio of molybdenum to beryllium is from 1:1 to 1:50.

In some embodiments, the molar ratio of molybdenum to vanadium is from 1:0.25 to 1:0.40, the molar ratio of molybdenum to tellurium is from 1:0.07 to 1:0.20, the molar ratio of molybdenum to niobium is from 1:0.10 to 1:0.20, and the molar ratio of molybdenum to beryllium is from 1:1 to 1:50.

In some embodiments, the molar ratio of molybdenum to vanadium is from 1:0.30 to 1:0.35, the molar ratio of molybdenum to tellurium is from 1:0.10 to 1:0.17, the molar ratio of molybdenum to niobium is from 1:0.12 to 1:0.15, and the molar ratio of molybdenum to beryllium is from 1:1 to 1:50.

In some embodiments, the catalyst material includes less than 0.005 wt. % sulfur. For example, the catalyst material can include less than 0.003 wt. % sulfur. In some embodiments, the catalyst material includes from 0.01 wt. % to 1.0 wt. % nitrogen. For example, the catalyst material can include from 0.1 wt. % to 0.3 wt. % nitrogen. In some embodiments, the catalyst material includes from 25 wt. % to 35 wt. % oxygen. For example. the catalyst material can include from 27 wt. % to 33 wt. % oxygen.

As discussed further below, the catalyst material can be formed by combining a catalyst, such as a mixed metal oxide catalyst, e.g., a mixed metal oxide catalyst including molybdenum, vanadium, tellurium, niobium, and oxygen, with an additive containing beryllium, such as beryllium oxide (BeO).

### Properties of Catalyst Materials

The molar ratio of Mo:Be in the catalyst materials described herein affects the activity of the catalyst materials. By activity, we mean the ability of the catalyst material to increase the rate of reaction. When the molar ratio of Mo:Be is below a threshold (meaning the molar amount of Be is higher than a threshold amount relative to the molar amount of Mo), the activity of the catalyst material is lower than the activity of the corresponding catalyst. When the molar ratio of Mo:Be exceeds the threshold (meaning the molar amount of Be is lower than a threshold amount relative to the molar amount of Mo), the activity of the catalyst material is higher than the activity of the corresponding catalyst.

The threshold ratio can be a molar ratio of Mo:Be of 1:8. When the molar ratio of Mo:Be is less than 1:8, e.g., 1:10, 1:12, 1:15, 1:18, 1:20, 1:25, 1:30, 1:35, 1:40, 1:45, or 1:50, the activity of the catalyst material is lower than the activity of the corresponding catalyst. When the molar ratio of Mo:Be is greater than 1:8, e.g., 1:7, 1:6, 1:5, 1:4, 1:3, 1:2, or 1:1, the activity of the catalyst material is higher than the activity of the corresponding catalyst.

The activity of a material can be quantified by the 35% conversion temperature of the material, where a higher 35% conversion temperature indicates a lower activity. When the molar ratio of Mo:Be is below the threshold value (e.g., when the molar ratio of Mo:Be is between 1:8 and 1:50), the 35% conversion temperature of the catalyst material is higher than the 35% conversion temperature of the corresponding catalyst. When the molar ratio of Mo:Be exceeds the threshold value (e.g., when the molar ratio of Mo:Be is between 1:1 and 1:8), the 35% conversion temperature of the catalyst material is lower than the 35% conversion temperature of the corresponding catalyst.

When the molar ratio of Mo:Be is below the threshold value, the 35% conversion temperature of the catalyst material can be between about 400° C. and 410° C., e.g., 400° C., 402° C., 404° C., 406° C. , 408° C., or 410° C. When the molar ratio of Mo: Be exceeds the threshold value, the 35% conversion temperature of the catalyst material can be between about 370° C. and 390° C., e.g., 370° C. , 372° C. , 374° C. 376° C., 378° C., 380° C., 382° C. , 384° C., 386° C., 388° C., or 390° C.

As used in this disclosure, the phrase “35% conversion temperature” refers to the temperature at which 35% of ethane in a gas stream is converted to a product other than ethane using an microreactor unit (MRU) and test conditions described below. Conversion of the feed gas is calculated as a mass flow rate change of ethane in the product compared to the feed ethane mass flow rate using the following formula:

$$C = ( \frac{2 * {X}_{{C}_{2} {H}_{4}} + 2 * {X}_{{CH}_{3} COOH} + {X}_{{CO}_{2}} + {X}_{CO}}{2 * {X}_{{C}_{2} {H}_{4}} + 2 * {X}_{{C}_{2} {H}_{6}} + 2 * {X}_{{CH}_{3} COOH} + {X}_{{CO}_{2}} + {X}_{CO}} ) * 100 \%$$

where C is the percent of feed gas that has been converted from ethane to another product (i.e., ethane conversion) and X is the molar concentration of the corresponding compound in the gaseous effluent exiting the reactor. The ethane conversion is then plotted as a function of temperatures to acquire a linear algebraic equation. The linear equation for ethane conversion is solved to determine the temperature in which the ethane conversion is 35% (i.e. the 35% conversion temperature).

The catalyst materials described here have a high selectivity to ethylene. For instance, the selectivity of the catalyst materials to ethylene can be between about 90% and about 100% at a temperature of between about 350° C. and about 500° C. , e.g., between about 95% and about 99%, e.g., between about 97% and about 98%, such as 95%, 97%, 98%, or 99%. The selectivity of the catalyst materials to ethylene can be substantially independent of the amount of Be present in the catalyst materials.

As used in this disclosure, the phrase “selectivity to ethylene” refers to the percentage on a molar basis of converted or reacted ethane that forms ethylene. An oxidative dehydrogenation catalyst's selectivity to ethylene can be determined using an MRU as discussed above. An oxidative dehydrogenation catalyst's selectivity to ethylene can be determined using to the following equation:

$${S}_{{C}_{2} {H}_{4}} = ( \frac{2 * {X}_{{C}_{2} {H}_{4}}}{2 * {X}_{{C}_{2} {H}_{4}} + 2 * {X}_{{CH}_{3} COOH} + {X}_{{CO}_{2} + {X}_{CO}}} ) * 100 \%$$

where S_{C2H4} is the selectivity to ethylene and X is the molar concentration of the corresponding compound in the gaseous effluent exiting the reactor. Notably, the selectivity to ethylene is determined at the 35% conversion temperature (discussed below), unless otherwise indicated. As such, after the 35% conversion temperature is determined, the above equation for selectivity is solved using the corresponding values for X_{C2H4} , X_{CO2}, and X_{CO} at the 35% conversion temperature.

Oxidative dehydrogenation of ethane may also result in production of various other byproducts including maleic acid, propionic acid, ethanol, and acetaldehyde. The amounts of these byproducts are insignificant, forming less than 0.1 mol % of the product, and are therefore not included in the calculations for conversion and selectivity.

### MRU Testing

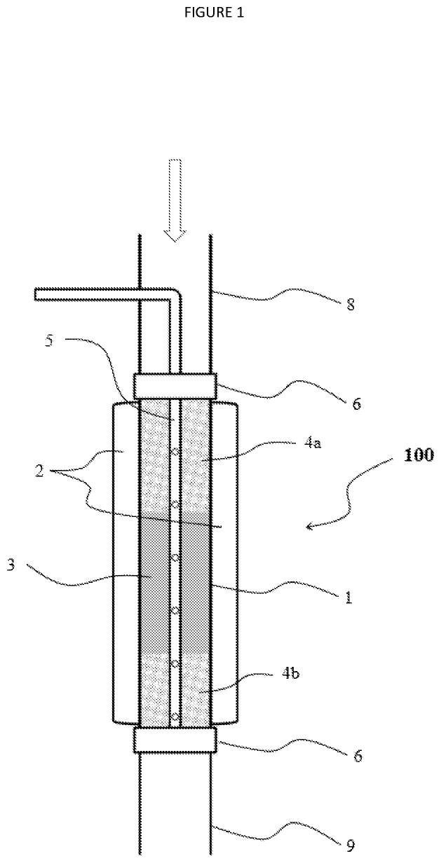

The ability of catalyst materials described herein to participate in the oxidative dehydrogenation of ethane can be tested in a microreactor unit (MRU) 100, shown in cross-section in FIG. 1. MRU 100 consists of a vertically oriented reactor tube 1 formed from stainless-steel SWAGELOK® tubing having an outer diameter of 0.5 inches, an inner diameter of 0.4 inches, and a length of 15 inches, surrounded by a two-zone electrical heater 2 or tube furnace and connected to tubing above and below via SWAGELOK connections 6. A catalyst bed 3 (gray shading) containing the catalyst, or catalyst material, situated at or near the middle of the reactor tube (along the length) is secured in place by packing 4 comprising glass wool bordering the upper (4a) and lower (4b) boundaries of the catalyst bed (hatched shading). A 6-point WIKA Instruments Ltd. K-type thermocouple 5 having an outer diameter of 0.125 inches inserted through the center of and along the length of the reactor tube 1 was used to measure the temperature within the catalyst bed. The temperature input from thermocouple 5 is used to control the power output to the electrical heater 2 in order to control the temperature inside the reactor. The 6-points, indicated by hollow circles, are spread along the length of reactor tube 1, with points 3 and 4 situated within the catalyst bed 3 and used as the reaction temperature controlling points. Points 1, 2, 5, and 6 of thermocouple 5 may be used for monitoring of feed heating and product quenching performance. Two feed gas lines are attached to the reactor (not shown), with one line dedicated to high purity nitrogen purge gas and the other line connected for introducing a process feed gas (indicated by hollow arrow). A room temperature stainless steel condenser is located downstream of the reactor to collect water/acetic acid condensates. The gas product flow may be allowed to either vent or may be directed to a gas chromatography (GC; Agilent 6890N Gas Chromatograph, Using Chrom Perfect—Analysis, Version 6.1.10 for data evaluation) via a sampling loop (not shown).

The catalyst bed is prepared by mixing 1.96 g of catalyst or catalyst material with quartz sand in an approximate 1:1 volume ratio, resulting in a total volume of about 3 mL.

For the MRU testing, a pre-mixed process feed gas comprising 36 mol. % ethane, 18 mol. % oxygen, and 46 mol. % nitrogen is fed to the reactor, passing from upper tubing 8, through catalyst bed 3 where conversion occurs, with effluent gas exiting through lower tubing 9. The molar ratios of the feed gas may vary by up to +1 mol. % during testing. The pre-mixed feed may be prepared using gas blending equipment and calibrated mass flow controllers (not shown). An outlet pressure of 0 psig is to be maintained, and the flow of the pre-mixed feed gas controlled in order to achieve a constant weight hourly space velocity (WHSV) of 2.90^{h-1}, where WHSV is defined as mass flow of feed gas to the reactor divided by the weight of the catalyst in the catalyst bed. The gas exiting the reactor is analyzed by GC to determine the percent of various hydrocarbons (e.g., ethane and ethylene) and optionally other gases such as O₂, CO₂, and CO and acetylene, with the results being used to calculate conversion and selectivity as defined above. Temperature is monitored in real-time at all 6 points, with the average of points 3 and 4 (which are within the catalyst bed) providing the temperatures used for plotting conversion versus temperature.

### Synthesis and Use of Catalyst Materials

In some examples, a catalyst material can be synthesized by combining a catalyst with an additive including beryllium. The catalyst can be a mixed metal oxide including molybdenum, vanadium, tellurium, niobium, and oxygen. The additive including beryllium can be a beryllium oxide.

In some embodiments, the catalyst includes a mixed metal oxide having the empirical formula:

Mo_{1.0}V_{0.12-0.49}Te_{0.01-0.30}Nb_{0.01-0.30}O_{d}

where d is a number to satisfy the valence of the oxide.

In some embodiments, the catalyst includes a mixed metal oxide having the empirical formula:

Mo_{1.0}V_{0.20-0.45}Te_{0.05-0.25}Nb_{0.05-0.25}O_{d}

where d is a number to satisfy the valence of the oxide.

In some embodiments, the catalyst includes a mixed metal oxide having the empirical formula:

Mo_{1.0}V_{0.25-0.40}Te_{0.07-0.20}Nb_{0.10-0.20}O_{d}

where d is a number to satisfy the valence of the oxide.

In some embodiments, the catalyst includes a mixed metal oxide having the empirical formula:

Mo_{1.0}V_{0.30-0.35}Te_{0.10-0.17}Nb_{0.12-0.15}O_{d}

where d is a number to satisfy the valence of the oxide.

In a process for making a catalyst material an additive containing beryllium (e.g., BeO) is combined with a catalyst, such as a mixed metal oxide catalyst, e.g., a mixed metal oxide including molybdenum, vanadium, tellurium, niobium, and oxygen. The catalyst and additive containing beryllium can be combined in a weight ratio of less than 99:1, e.g., between 92:8 and 40:60, e.g., 92:8, 80:20, 70:30, 60:40, 50:50, 40:60, 30:70, or another amount. Distilled water is added to the mixture of the catalyst and the additive containing beryllium to form an aqueous mixture, such as a slurry. The slurry is stirred and heated to allow the water to slowly evaporate, inducing a hydrothermal reaction that results in formation of a paste. The slurry can be stirred at a temperature of between 90° C. and 110° C., e.g., 90° C., 92° C., 94° C., 96° C. , 98° C., 100° C. 102° C. , 104° C., 106° C., 108° C., or 110° C. The slurry can be stirred for an amount of time sufficient to induce formation of the paste, such as for between 30 minutes and 2 hours, e.g., 30 minutes, 45 minutes, 1 hour, 75 minutes, 90 minutes, 105 minutes, or 2 hours.

The paste is dried at elevated temperature, e.g., at a temperature of between 70° C. and 100° C. e.g., 70° C. , 75° C., 80° C., 85° C., 90° C. , 95° C., or 100° C. The paste can be dried for several hours, e.g., overnight, e.g., for between 2 hours and 24 hours, e.g., 2 hours, 4 hours, 6 hours, 8 hours, 10 hours, 12 hours, 14 hours, 16 hours, 18 hours, 20 hours, 22 hours, or 24 hours. The dried paste is then calcined to form the catalyst material. For instance, the paste can be calcined, e.g., in an oven or a muffle furnace, at a temperature of about between 330° C. and 380° C. , e.g., 330° C. 335° C., 340° C. , 345° C., 350° C., 355° C. , 360ºC. 365° C., 370° C., 375° C., or 380° C. The calcining process can proceed for 1-3 hours, e.g., 1 hour, 1.5 hours, 2 hours, 2.5 hours, or 3 hours.

The formation of catalyst materials by mixing an additive containing Be with a catalyst can facilitate the tuning of the activity of the catalyst while leaving the selectivity of the material to ethylene generally high and unaffected. For instance, to tune the activity of a catalyst, Be is incorporated into the catalyst by either adding a first quantity of Be to the catalyst to form a first catalyst material having an activity higher than an activity of the catalyst, or adding a second quantity of Be to the catalyst to form a second catalyst material having an activity of the second catalyst material lower than the activity of the catalyst. The selectivity of the two catalyst materials to ethylene is substantially the same, e.g., within 5%, within 2%, within 1%, or within 0.5%.

In some examples, the catalyst materials described here are used in a reactor for oxidative dehydrogenation of alkanes, such as ethane, for production of ethylene. A feed material, which can include ethane, is received into the reactor, and a product, such as ethylene, is output from an outlet of the reactor. For instance, the reactor can be a fixed bed catalytic reactor, including, but not limited to, shell-and-tube type reactors. A shell-and-tube reactor with molten salt cooling capabilities is also contemplated.

The reactor can include multiple sections, each of which operates at a different temperature. The sections may be enclosed in a single reactor, or they may be spread across two or more reactors, each reactor comprising one or more sections. Operating the sections at different temperatures can facilitate energy recovery from the oxidative dehydrogenation reaction. For instance, the first section can be operated at the lowest temperature, e.g., at 350° C., resulting in production of lowest quality energy. The second section can be operated at a higher temperature, e.g., between 400° C. and 500° C. The increase in reaction temperature along the sections gives rise to the capability to produce high pressure steam, thereby enabling some of the energy from the oxidative dehydrogenation reaction to be recovered as mechanical energy. With the catalyst materials described here, the activity of the catalyst materials can be tailored to each individual section without adversely affecting the selectivity of the oxidative dehydrogenation process. For instance, a catalyst material having a Mo:Be ratio below the threshold value in the second section operating at a higher temperature reactor section allows the functionality of the oxidative dehydrogenation process even at high temperatures to be maintained. The lower activity of these catalyst materials can allow operation at higher temperatures without compromising selectivity.

In some examples, the catalyst materials described here can be formed in conjunction with a support material, such as alumina, zirconia, titania, zeolites, or another suitable support material. For instance, the catalyst materials can be mixed together with a support material, e.g., by dry or wet mixing, and formed into a shape for use in the reactor, such as by extruding, pressing, or another suitable technique.

The presence of Be in the catalyst materials described here can facilitate the extrusion or pressing of the catalyst materials in conjunction with the support material because beryllium-containing oxides tend to be softer than the mixed metal oxides of the corresponding catalysts. The presence of Be in the catalyst material can also contribute to enhancing the strength of the final material that is the combination of the catalyst material and the support material, e.g., as compared to a material that is a combination of the corresponding catalyst and a support material.

### EXAMPLES

The following examples describe the synthesis of samples of catalysts and catalyst materials and the characterization according to composition (ICP-MS, XRD, SEM) and performance (conversion and selectivity to ethylene). The results demonstrate the high selectivity of beryllium-containing catalyst materials and the effect of the Mo:Be ratio on the activity of the catalyst materials, as evidenced by the 35% conversion temperature.

### Catalyst Synthesis

### Catalyst 1.1

Synthesis of a mixed metal oxide catalyst (referred to as catalyst 1.1) was as follows:

To a 400 ml beaker was charged 70.09 g of VOSO₄·3.35H₂O and 100 mL of distilled water. This mixture was left to stir for 30 minutes in a 60° C. water bath with a stir speed of 300 rpm, becoming a clear, electric blue solution.

To a 2 L round bottom flask was charged 96.1334 g of (NH₄)₆TeMo₆O₂₄·7H₂O and 300 mL of distilled water to form a TeMoOₓ solution. This mixture was stirred at 300 rpm for 30 minutes in a 60° C. warm water bath. The stirred VOSO₄ solution was added dropwise using a dropper funnel to the turbid, white TeMoOₓ solution over 30 minutes. The resulting TeMoVOₓ solution was stirred for 15 minutes. 192.20 g of a NbO(C₂O₄H)₃ (aq) solution (0.356 mmol Nb/g solution) was added dropwise to the stirred TeMoVOₓ solution over 20 minutes.

The resulting solution was transferred to a 2 L PARR reactor (Parr Instrument Company, Moline, IL), which was scaled, evacuated, and backfilled with nitrogen three times. The PARR reactor was left under 15 bar of nitrogen gas, connected to a back-pressure regulator, kept sealed, and left to stir at 300 rpm overnight at room temperature. The 15-bar nitrogen was bubbled through the tubing connecting the PARR reactor to the back-pressure regulator and condenser. Pressure was dialed into 160 psi on the backpressure regulator. Reactor exterior temperature was set to 185° C. and reactor interior temperature was set to 165° C. After 24 hours of heating the heat was removed and the reactor was left to cool to room temperature. The following day the reactor was depressurized and the contents were removed and filtered through a 150 mm Buchner funnel using 4 Whatmann #2 filter papers. The filter cake was a purple/brown color and the filtrate was a blue color. The filter cake was rinsed with distilled water until the filtrate ran clear, using approximately 1 L of distilled water. The filter cake was dried in a 90° C. oven for 60 hours. The catalyst was ground and calcined in the quartz reactor unit at 600° C. for 2 hours under nitrogen.

### Catalyst 1.2

Synthesis of a mixed metal oxide catalyst (referred to as catalyst 1.2) was as follows:

To a vessel was charged 10 L of distilled water, and the water was heated to 65° C. To this vessel was also charged 1102.0 grams of oxalic acid (C₂H₂O₄₍ₛ₎; 12.240 mol), which dissolved quickly with stirring to form a clear, colorless solution. To the 65° C. aqueous oxalic acid solution was then charged 656.3 grams of diniobium pentoxide hydrate (Nb₂O₅·xH₂O₍ₛ₎). The weight of diniobium pentoxide hydrate was weighed based on an 80% weight of Nb₂O₅ with MW of 265.81 g/mol, which is 525.06 g (1.975 mol) of Nb₂O₅. This addition formed a white suspension. The vessel opening was rinsed with 1 L of distilled water rinsing the residual powders into the solution, producing a total volume of 11 L. The 11 L of aqueous, white suspension was left to heat and stir at 65° C. for 24 hours to 72 hours. After the 24-72 hours of heating at 65° C., the solution was cooled down to ambient conditions. The solution of H₃[NbO(C₂O₄)₃]_{(aq)} was clear and colorless with only small amounts of white insoluble matter at the bottom of the vessel in the absence of mixing. Once cooled to room temperature the solution can be stored for extended periods of time before use.

To another vessel was charged 6 L of distilled water, and the water was heated to 60° C. 1054.6 grams of telluric acid (Te(OH)₆₍ₛ₎; 4.593 mol) was added to the 60° C. distilled water. The telluric acid dissolved easily upon stirring to form a clear and colorless solution. The vessel opening was rinsed with 1 L of distilled water to rinse the powders into solution, producing a final volume of 7 L. The 60° C., 7 L Te(OH)_{6(aq)} solution was cooled down to room temperature and held for the following steps.

To a jacketed glass reactor, 16 L of distilled water was added and heated using a circulation bath and silicone oil. The 16 L of distilled water was heated to 30-35° C. To the jacketed vessel was then charged 4865.0 grams of ammonium molybdate tetrahydrate ((NH₄)₆Mo₇O₂₄·4H₂O₍ₛ₎; 3.934 mol), which dissolved with stirring to form a turbid white solution. The vessel opening was rinsed with 1 L of distilled water to rinse any residual solids into solution, producing a total volume of 17 L.

The entire 7 L TeOH6 solution was transferred at ambient temperature to the jacketed vessel, which contained the stirred solution of ((NH₄)₆MoO₂₄·4H₂O_{(aq)}) turbid solution, at an addition rate of 412 mL/min to form a clear and colorless solution (referred to as the “MoTe solution”). The vessel that contained the telluric acid was rinsed with 1 L of distilled water, and the rinse water was transferred to the jacketed glass reactor. The MoTe solution was heated to 80° C. and the pH was adjusted to 7.40-7.60 using 1680-2000 grams (calculated 1.85-2.20 L at density of 0.91 g/cm³) of 28-30% ammonium hydroxide solution. The pH 7.50 MoTe solution was stirred at 80° C. for one hour, after which the pH of the MoTe solution was adjusted from 7.50 to 4.9-5.1 using 1270-1550 grams (calculated 0.69-0.84 L at density of 1.85 g/cm3) of 95-08% sulfuric acid.

The MoTe solution, now an aqueous ammonium molybdotellurate ((NH₄)₆Mo₆TeO_{24(aq)}), was transferred to a hydrothermal reactor preheated to 60° C. The glass reactor was rinsed with 2 L of distilled water and the rinse water was transferred to the 60° C. pre-heated hydrothermal reactor. The 60-80° C. MoTe solution was stirred via agitator inside the high-pressure hydrothermal reactor.

To a separate glass vessel was charged 11 L of distilled water. The water was heated to 60° C. To the water was charged 4023.5 grams of vanadyl sulfate hydrate (VOSO₄·3.35H₂O (s); 18.10 mol). The powder dissolved with vigorous stirring, forming a clear, blue solution. The vessel opening was rinsed with 1 L of distilled water to rinse residual powders into solution, producing a total volume of 12 L. The 60° C., 12 L VOSO_{4(aq)} solution was held at 60° C. for additional steps.

To the ammonium molybdotellurate solution stirred in the high-pressure hydrothermal reactor at 55-65° C., was charged the entire volume of the 60° C. vanadyl sulfate solution at an addition rate of 367 ml/min. The vanadyl sulfate vessel was rinsed with 2 L of distilled water and the rinse water was transferred to the high-pressure hydrothermal vessel. The resulting black solution was stirred for 30 minutes at 55-60° C.

After 30 minutes, the entire volume of room temperature niobium oxalate solution was transferred to the MoTeV solution in a stirred reactor autoclave, 55-60° C., at an addition rate of 183 mL/min to form a purple slurry. The niobium oxalate vessel was rinsed with 2 L of distilled water and the rinse water was transferred to the high-pressure hydrothermal reactor. After the addition of all reagents, the reactor was heated to 160-165° C.

The slurry inside the reactor was heated to 160-165° C., while the pressure was maintained at 95-105 psi with the use of a back-pressure regulator built into the reactor head. Custom heating mantles, insulation and temperature programming control were used to heat the reactor slurry to 160-165° C., without exceeding 185° C. at the metal surface of the reactor. The slurry in the reactor was heated for 24-48 hours. The reaction was cooled by removing heat and insulation for 17-20 hours. The slurry was stirred during cool down at the same rate as the hydrothermal reaction (100 rpm).

The solids from the hydrothermal reaction were filtered and recovered. The solids separated from the mother liquor are herein referred to in this example as pre-catalyst. After the pre-catalyst was washed and dried at 90° C. in a drying pans for 3-5 days, the solids were crumbly and friable. This material, herein referred to as uncalcined catalyst in this example, was ground to 125-500 μm size range. This ground, uncalcined catalyst was dried in drying boats by holding at 250° C. for 6 hours under air to reduce the moisture to <2%, yielding 5.9-6.3 kg of uncalcined catalyst before allowing to naturally cool at room temperature. The uncalcined catalyst was calcined in a quartz reactor under nitrogen. The quartz reactor was ramped to 600° C. at 1.6° C. per minute and held at 600° C. for 2 hours. The quartz reactor was then cooled to room temperature before removing the catalyst.

### Catalyst Material 2.1

Synthesis of a catalyst material (referred to as catalyst material 2.1), starting with 8 wt. % BeO and 92 wt. % catalyst 1.1, was as follows:

To a 100 ml beaker was charged 0.8010 g of beryllium oxide (Sigma-Aldrich, Lot #MKBW4990V) and 9.2016 g of catalyst 1.1. Next, the beaker was charged with 10 mL of distilled water. The mixture containing water, catalyst 1.1, and beryllium oxide was stirred with an overhead agitator using a glass rod and a 0.5 inch stir blade. The mixture was stirred at 100 rpm and sat while boiling the water off using a hotplate set to 100° C. for 1 hour until the slurry became a paste. The paste, a pale purple color, was dried overnight at 90° C. The paste was then calcined at 350° C. for 2 hours with a 30-minute ramp time to form catalyst material 2.1. The composition of the catalyst material was analyzed by ICP-MS and the activity and selectivity of the catalyst material was tested in a microreactor unit.

### Catalyst Material 2.2

Synthesis of a catalyst material (referred to as catalyst material 2.2) starting with 20 wt. % BeO and 80 wt. % catalyst 1.2, was as follows:

To a 100 ml beaker was charged 2.04 g of beryllium oxide, 8.04 g of catalyst 1.2. and 10 mL of distilled water. An overhead agitator was set up with a glass stir rod and a ½″ Teflon® stir blade. Subsequently, an oil bath was used to heat the beaker, the oil bath being set to 100° C. and stirred at 100 rpm until the mixture became a paste. The paste was then dried in a 90° C. oven overnight. The dried paste was then calcined at 350° C. in oven for 2 hours with a 30-minute ramp cycle to form catalyst material 2.2. The catalyst material was then analyzed by ICP-MS as disclosed herein and tested in a microreactor unit (MRU).

### Catalyst Material 2.3

Synthesis of a catalyst material (referred to as catalyst material 2.3) starting with 40 wt. % BeO and 60 wt. % catalyst 1.2, was as follows:

To a 100 ml beaker was charged 6.0 g of Catalyst 1.2, 4.0 g of beryllium oxide and 15 mL of deionized water. The mixture was stirred manually to make a slurry. Subsequently, the beaker was heated in an oil bath at 100° C. and an overhead stirrer was used to stir the mixture at approximately 90 rpm. The mixture was stirred until a paste formed which took approximately 45 minutes. The beaker containing paste was placed in an oven at 90° C. to dry overnight. Subsequently, the beaker was placed into a muffle furnace at 350° C. for 2.5 hours. After, the 2.5 hours the muffle furnace was turned off and the beaker with the catalyst material was allowed to cool overnight.

### Catalyst Material 2.4

Synthesis of a catalyst material (referred to as catalyst material 2.4) starting with 60 wt. % BeO and 40 wt. % catalyst 1.2, was as follows:

To a 100 mL beaker was charged 6.0281 g of beryllium oxide (Sigma Aldrich, Lot # MKBW4990V) and 4.0618 g of catalyst 1.2. 10 mL of distilled water was charged to the beaker producing a light purple slurry. An overhead agitator was assembled using a glass stir shaft and a ½″ Teflon stir blade. The slurry was stirred at 106 rpm. An oil bath was used to heat the beaker while being stirred with an overhead agitator. The temperature of the oil bath was set to 100° C. The slurry was left to stir until the slurry had evaporated into a paste, this process took 30 minutes. The light purple paste was removed from the oil bath and placed in a 90° C. oven to dry overnight. The light purple paste became a light purple chunky powder. The chunks were broken up to form a consistent light purple powder. This powder was calcined in an air muffle furnace for two hours, with a 30-minute ramp time to form catalyst material 2.4.

### Catalyst Material 2.5

Synthesis of a catalyst material (referred to as catalyst material 2.5) starting with 3.2 wt. % BeO and 96.8 wt. % catalyst 1.2, was as follows:

To a 100 ml beaker was charged 9.68 g of Catalyst 1.2, 0.32 g of beryllium oxide and 15 mL of deionized water. The mixture was stirred manually to make a slurry. Subsequently, the beaker was heated in an oil bath at 100° C. and an overhead stirrer was used to stir the mixture at approximately 100 rpm. The mixture was stirred until a paste formed which took approximately 45 minutes. The beaker containing paste was placed in an oven at 90° C. to dry overnight. Subsequently, the beaker was placed into a muffle furnace at 350° C. for 2 hours. After, the 2 hours the muffle furnace was turned off and the beaker with the catalyst material was allowed to cool overnight.

### Catalyst Material Characterization

### Calculation of Molar Ratios by ICP-MS.

Samples were solubilized for ICP-MS analysis via digestion in a 50 wt. % oxalic acid solution. Calibration of the ICP-MS was performed using external standards matched to the matrix of the sample and the curves were calculated after subtracting the reagent blank. Several elements (Li, Sc, Y, In. Tb, and Bi) served as internal standards and were mixed continuously through online addition to monitor and compensate for signal drift. The results were generally reported as μg/g (ppmw) or μg/L (ppbv).

To calculate the molar ratios of elements in the catalyst or catalyst material, the following equation was used:

$${R}_{E l} = \frac{{C}_{El} / {M}_{el}}{{C}_{Mo} / 95.94}$$

where R_{EI} is the ratio number for the corresponding element (e.g., Mo, V, Nb, Te, Be), C_{EI} is the weight concentration (e.g., wt.-ppm) of the corresponding element, M_{EI} is the molar mass (e.g., in g/mol) of the corresponding element, C_{Mo} is the weight concentration (e.g., wt.-ppm) of molybdenum (Mo) in the corresponding catalyst, and 95.94 is the molar mass of Mo in g/mol. Application of the above equation provides the elemental ratios of the elements in the catalyst material, with the ratio number for Mo in this calculation being assigned to be 1. In some examples, a different element in the catalyst material can be assigned the ratio number of 1, which will change the ratio numbers for all the other elements.

The data in Table 1 summarizes the molar ratios of molybdenum, vanadium, niobium, tellurium, and beryllium (for catalyst materials) for each of the samples prepared (catalysts and catalyst materials) as determined by ICP-MS analysis.

TABLE 1
Molar Ratios of Samples Prepared

| Sample | Mo | V | Nb | Te | Be |
|---|---|---|---|---|---|
| Catalyst 1.1 | 1 | 0.32 | 0.20 | 0.15 | 0.00 |
| Catalyst 1.2 | 1 | 0.29 | 0.21 | 0.16 | 0.00 |
| Catalyst Material 2.1 | 1 | 0.33 | 0.11 | 0.16 | 0.57 |
| Catalyst Material 2.2 | 1 | 0.33 | 0.11 | 0.14 | 1.82 |
| Catalyst Material 2.3 | 1 | 0.29 | 0.21 | 0.16 | 8.35 |
| Catalyst Material 2.4 | 1 | 0.29 | 0.21 | 0.16 | 18.79 |

### Pore Structure

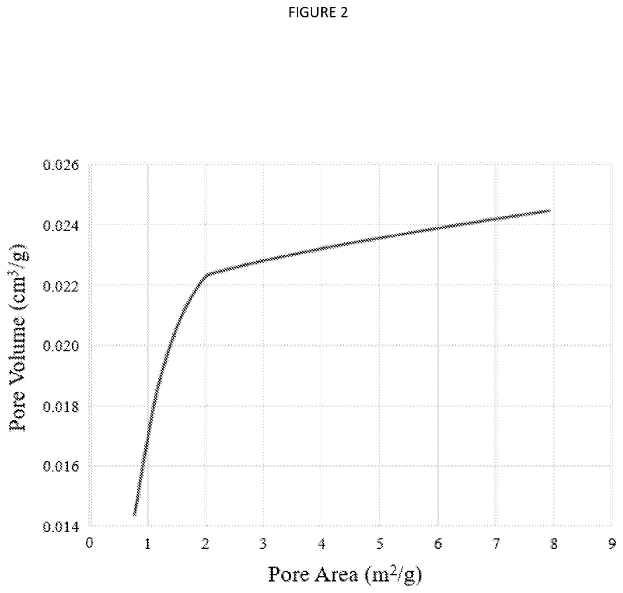

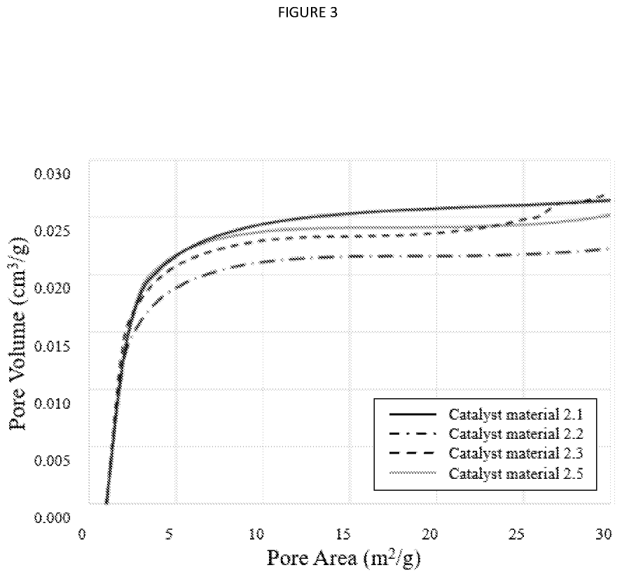

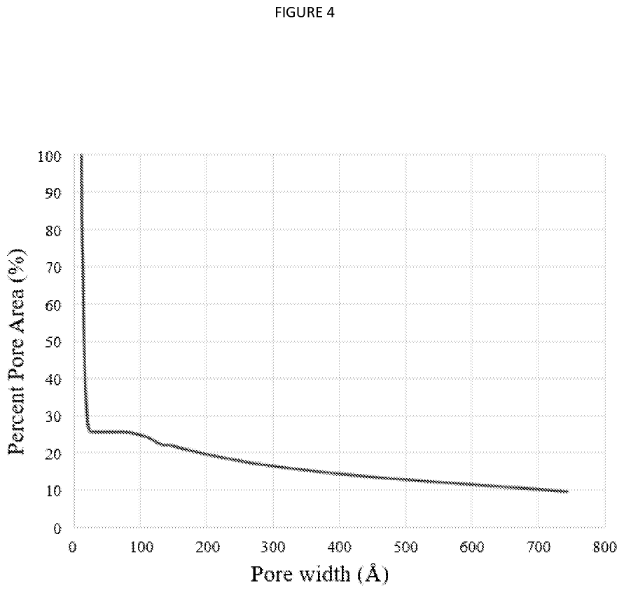

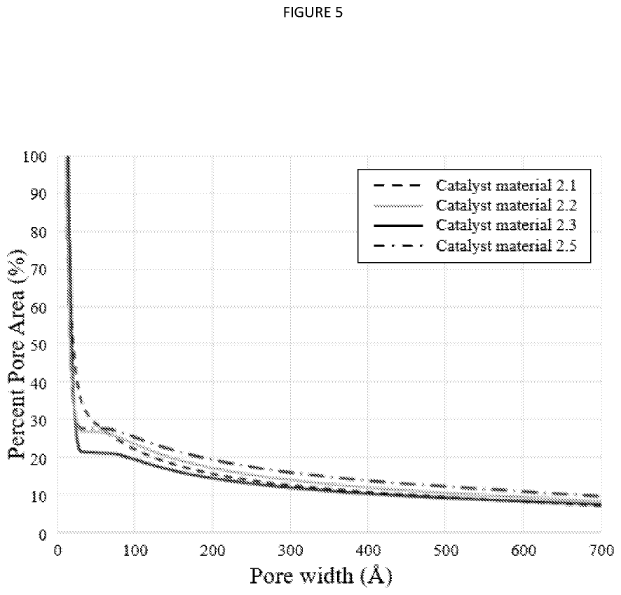

BET (Brunauer-Emmett-Teller) analysis was performed on catalyst 1.2 and catalyst materials 2.1-2.3, and a catalyst material 2.5 having 3.2 wt. % BeO. FIGS. 2 and 3 show the pore area vs. pore volume for catalyst 1.2 and for catalyst materials 2.1-2.3 and 2.5, respectively, as determined by BET analysis. FIGS. 4 and 5 show the percent pore area vs. pore width for catalyst 1.2 and for catalyst materials 2.1-2.3 and 2.5, respectively, as determined by BET analysis. The data indicates that addition of beryllium to the catalyst did not result in significant changes to the relationship between pore area and pore volume.

The surface area and pore volume for catalyst 1.2 and catalyst materials 2.1-2.3 and 2.5 were determined using a Barrett-Joyner-Halenda (BJH) analysis. A summary of the BJH data is given in Table 2. The BJH data indicate that both the surface area and the pore volume of the catalyst materials are unchanged relative to those of the catalyst.

TABLE 2
Surface Area and Pore Volumes

| Material | Surface Area (m²/g) | Pore Volume (cm³/g) |
|---|---|---|
| Catalyst 1.2 | 6 | 0.02 |
| Catalyst Material 2.1 | 9 | 0.03 |
| Catalyst Material 2.2 | 8 | 0.02 |
| Catalyst Material 2.3 | 8 | 0.02 |
| Catalyst Material 2.5 | 7 | 0.02 |

### Particle Size Analysis

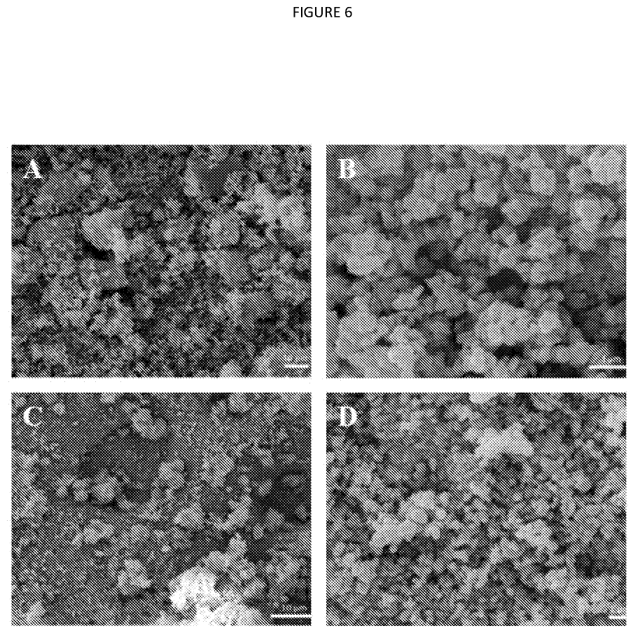

Scanning electron microscopy (SEM) was used to determine the size distribution and agglomerate size distribution for catalyst 1.2, catalyst materials 2.1-2.3 and 2.5, as well as for BeO reagent without any active catalyst. FIG. 6 shows SEM images of catalyst material 2.5 at 1,000× magnification (A) and 20,000× magnification (B), and SEM images of catalyst material 2.3 at 1,000× magnification (C) and 20,000× magnification (D). SEM images reveal that the catalyst material including beryllium oxide was topologically similar to the catalyst without the addition of beryllium oxide.

The particle size distribution and agglomerate size distribution data, as determined using SEM, are shown in Tables 3 and 4, respectively. No difference in average particle size was observed between the baseline catalyst, beryllium oxide, and the catalyst materials. The median particle size for the baseline catalyst, catalyst materials, and beryllium oxide was 0.25 μm.

TABLE 3
Particle Size Distribution

| | BeO | Min. size | Max. size | Median size |
|---|---|---|---|---|
| Sample | (wt. %) | (μm) | (μm) | (μm) |
| Catalyst 1.2 | — | 0.07 | 4.71 | 0.24 |
| Catalyst Material 2.1 | 8 | 0.06 | 1.25 | 0.31 |
| Catalyst Material 2.2 | 20 | 0.06 | 1.25 | 0.25 |
| Catalyst Material 2.5 | 3.2 | 0.10 | 0.77 | 0.33 |
| BeO | 100 | 0.05 | 1.33 | 0.20 |

TABLE 4
Agglomerate Size Distribution

| | BeO | Min. size | Max. size | Median size |
|---|---|---|---|---|
| Sample | (wt. %) | (μm) | (μm) | (μm) |
| Catalyst 1.2 | — | 0.07 | 4.71 | 0.24 |
| Catalyst Material 2.1 | 8 | 2.59 | 206.61 | 48.96 |
| Catalyst Material 2.2 | 20 | 6.86 | 206.61 | 48.96 |
| Catalyst Material 2.5 | 3.2 | 2.03 | 99.60 | 18.15 |
| BeO | — | 0.05 | 1.33 | 0.20 |

Catalyst 1.2 displayed a monomodal distribution of particle sizes within a range of 4 μm (not shown). The beryllium oxide also displayed a monomodal distribution of particle sizes, but within a range of 1.25 μm. When the baseline catalyst and beryllium oxide were combined into a catalyst material, the resulting particle size distribution was bimodal, with one mode having a range of 1 μm and the second mode having a range of 200 μm. These data indicate that the first mode reflects the size of the particles, and the second mode reflects the agglomerate size of catalyst-beryllium oxide mixtures in the catalyst materials.

### Crystal Structure by X-Ray Diffractometry

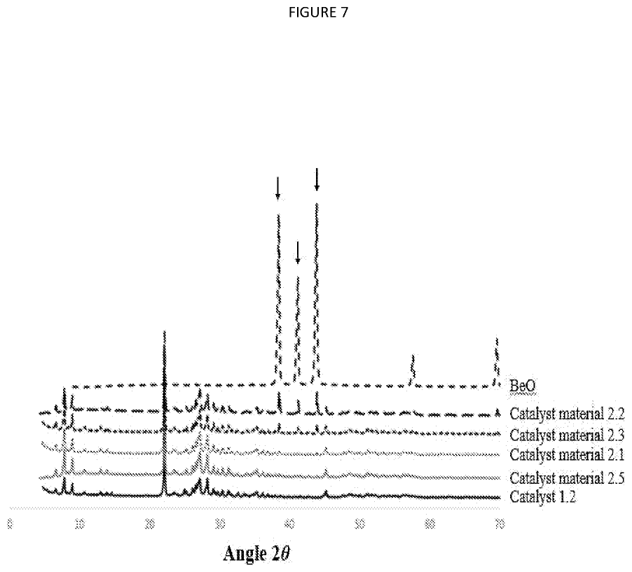

FIG. 7 shows X-ray diffraction (XRD) spectra for catalyst 1.2, catalyst materials 2.1-2.3 and 2.5, and beryllium oxide (overlapping). As the percentage of BeO increases, three peaks grow in the XRD spectrum with increasing intensity (indicated by arrows). These peaks are attributed to bromellite, which is the crystalline phase of beryllium oxide. These XRD data indicate that the beryllium oxide in the catalyst materials does not react in such a way to form a new crystal structure that interfaces the catalyst and the beryllium oxide, but rather the catalyst and the beryllium oxide co-exist as distinct crystal structures. The remainder of the elements in the catalyst materials form various crystal structures composed of molybdenum, vanadium, niobium and tellurium mixed metal oxides.

The crystalline phases of the catalyst materials form only a portion of the overall catalyst material. The difference between the overall catalyst material and the amount of crystalline material is the amorphous content of the catalyst material. The amount of amorphous content gives an indication of the active portion of the catalyst material. For instance, if the addition of an additive results in an increased crystalline or amorphous phase and also results in increased catalysis performance, this indicates that the phase that was increased may be involved in catalysis.

The amount of amorphous content in both the active phase catalyst materials and the BeO material can be measured using XRD results. The expectation is that the amount of amorphous phase should correspond to the percentage of the amorphous phase of each component. For instance, if the active catalyst material had 10% amorphous phase and the BeO had 20% amorphous phase; and 95% of the mixture is active phase and 5% is BeO, then the total expected amorphous content is 10.5% (i.e., 0.95*10+0.05*20). This principle was applied to catalyst materials 2.1-2.3 and 2.5. Tables 5 highlights the measured and theoretical amorphous phase content for each of the catalyst materials, along with catalyst 1.2 and BeO for comparison. The data did not suggest any trend with respect to the addition of beryllium oxide to the active catalyst. All differences observed were within standard deviation of the measurement. The standard deviation for the instrument translates to 5 weight percent in the amorphous content.

TABLE 5
Amorphous and Crystalline Content

| | Amorphous | Crystalline | Theoretical | Differ- |
|---|---|---|---|---|
| | Content | Content | Amorphous | ence |
| Sample | (%) | (%) | Content (%) | (%) |
| Catalyst 1.2 | 10 | 90 | — | — |
| BeO | 19.37 | 80.63 | | |
| Catalyst Material 2.1 | 12 | 88 | 10.8 | −1.2 |
| Catalyst Material 2.2 | 9.4 | 90.6 | 11.9 | +2.5 |
| Catalyst Material 2.3 | 15.1 | 84.9 | 13.8 | +1.3 |
| Catalyst Material 2.5 | 16.7 | 83.3 | 10.3 | −6.40 |

### Performance Analysis

The samples prepared were subjected to testing to measure performance using an MRU as described above. Specifically, samples (both catalysts and catalyst materials) were loaded into the MRU and subjected to the oxidative dehydrogenation process conditions described (e.g. feed composition, pressure, WHSV) in order to determine the 35% conversion temperature and the selectivity to ethylene at that temperature.

### Discussion of Activity and Selectivity of Catalysts and Catalyst Materials

Table 6 summarizes the performance of each of the catalysts and catalyst materials that were tested using the MRU. Included for reference is the molar ratio of Mo to Be for each catalyst material (based on wt. % of starting Mo and Be used for synthesis) and the difference in the 35% conversion temperature between the catalyst material and the catalyst used in preparation of the corresponding catalyst material. The results indicate that the selectivity of the catalyst materials to ethylene remains high irrespective of the molar ratio of Mo:Be. The 35% conversion temperature of the catalyst materials depends strongly on the Mo:Be ratio, with a transition to a significantly higher 35% conversion temperature occurring between Catalyst 2.2 (Mo:Be ratio of 1:3.3) and Catalyst 2.4 (Mo:Be ratio of 1:18.8).

TABLE 6
Performance Sμmmary

| | | Mo:Be | 35% Conv. | Delta T | Selectivity |
|---|---|---|---|---|---|
| Catalyst | Catalyst | Molar Ratio | Temp (° C.) | (° C.) | (%) |
| Catalyst 1.1 | — | | 375.45 | — | 97.00 |
| Catalyst 1.2 | — | | 379.59 | | 94.59 |
| Catalyst Material 2.1 | Catalyst 1.1 | 1:0.6 | 373.02 | −2.43 | 96.00 |
| Catalyst Material 2.2 | Catalyst 1.2 | 1:1.8 | 386.19 | +6.6 | 94.83 |
| Catalyst Material 2.4 | Catalyst 1.2 | 1:18.8 | 404.15 | +24.56 | 95.45 |

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

### INDUSTRIAL APPLICABILITY

The present disclosure relates to a catalyst material, comprising a mixed metal oxide catalyst and a beryllium containing additive, useful for oxidative dehydrogenation of ethane. The activity of the catalyst can be tuned by varying the amount of additive without negatively impacting selectivity to ethylene.

1. A catalyst material comprising:
a catalyst comprising a mixed metal oxide comprising molybdenum (Mo), vanadium (V), tellurium (Te), and niobium (Nb); and
an additive comprising beryllium (Be);
wherein:
the molar ratio of Mo: V is between 1:0.12 and 1:0.49;
the molar ratio of Mo: Te is between 1:0.01 and 1:0.30;
the molar ratio of Mo:Nb is between 1:0.01 and 1:0.30; and
the molar ratio of Mo:Be being less than 1:1; and
wherein the molar ratios are determined by ICP-MS.

2. The catalyst material of claim 1, wherein the molar ratio of Mo:Be is between 1:1 and 1:8.

3. (canceled)

4. The catalyst material of claim 1, wherein the catalyst comprises a mixed metal oxide comprising Mo_{1.0}V_{0.12-0.49}Te_{0.01-0.30}Nb_{0.01-0.30}O_{d}, wherein d is a number to satisfy a valence of the mixed metal oxide.

5. The catalyst material of claim 1, wherein a 35% conversion temperature of the catalyst material is between 370° C. and 390° C.

6. (canceled)

7. The catalyst material of claim 1, wherein the molar ratio of Mo:Be is between 1:8 and 1:50.

8. (canceled)

9. The catalyst material of claim 1, wherein a 35% conversion temperature of the catalyst material is between 370° C. and 425° C.

10. The catalyst material of claim 1, wherein a selectivity of the catalyst material to ethylene is between 90% and 100%.

11.-13. (canceled)

14. The catalyst material of claim 1, wherein:
the molar ratio of Mo:V is between 1:0.20 and 1:0.45;
the molar ratio of Mo:Te is between 1:0.05 and 1:0.25; and
the molar ratio of Mo:Nb is between 1:0.05 and 1:0.25.

15. The catalyst material of claim 14, wherein:
the molar ratio of Mo:V is between 1:0.25 and 1:0.40;
the molar ratio of Mo:Te is between 1:0.07 and 1:0.20; and
the molar ratio of Mo:Nb is between 1:0.10 and 1:0.20.

16. The catalyst material of claim 15, wherein:
the molar ratio of Mo:V is between 1:0.30 and 1:0.35;
the molar ratio of Mo:Te is between 1:0.10 and 1:0.17; and
the molar ratio of Mo:Nb is between 1:0.12 and 1:0.15.

17. The catalyst material of claim 1, wherein the molar ratios of Mo:V, Mo:Te, and Mo:Nb are determined by a mass spectrometry analysis.

18. A method of making a catalyst material, the method comprising:
forming an aqueous mixture comprising:
a catalyst comprising a mixed metal oxide comprising Mo_{1.0}V_{0.12-0.49}Te_{0.01-0.30}Nb_{0.01-0.30}O_{d}, wherein d is a number to satisfy a valence of the mixed metal oxide; and
an additive comprising Be;
heating the aqueous mixture to form a paste; and
baking the paste to form the catalyst material.

19. The method of claim 18, wherein forming an aqueous mixture comprises forming an aqueous mixture wherein a weight ratio of the catalyst to the additive is less than 92:8.

20. (canceled)

21. The method of claim 18, wherein forming an aqueous mixture comprises forming an aqueous mixture comprising the catalyst and BeO.

22. The method of claim 18, comprising drying the paste.

23. (canceled)

24. The method of claim 18, wherein baking the paste comprises calcining the paste at a temperature of between 330° C. and 380° C.

25. (canceled)

26. A reactor system for generation of ethylene from ethane, the reactor system comprising:
a reactor vessel; and
a catalyst material disposed in the reactor vessel, the catalyst material comprising:
molybdenum (Mo);
vanadium (V), the molar ratio of Mo:V being between 1:0.12 and 1:0.49;
tellurium (Te), the molar ratio of Mo:Te being between 1:0.01 and 1:0.30;
niobium (Nb), the molar ratio of Mo:Nb being between 1:0.01 and 1:0.30; and
beryllium (Be), the molar ratio of Mo:Be being less than 1:1;
wherein molar ratios are determined using ICP-MS.

27. A method for generation of ethylene from ethane, the method comprising:
processing ethane in a reactor in an oxidative dehydrogenation process in the presence of a catalyst material, the catalyst material comprising:
molybdenum (Mo);
vanadium (V), the molar ratio of Mo:V being between 1:0.12 and 1:0.49;
tellurium (Te), the molar ratio of Mo:Te being between 1:0.01 and 1:0.30;
niobium (Nb), the molar ratio of Mo:Nb being between 1:0.01 and 1:0.30; and
beryllium (Be), the molar ratio of Mo:Be being less than 1:1;
wherein molar ratios are determined using ICP-MS.

28. The method of claim 27, comprising processing the ethane at a temperature between 400° C. and 500° C.

29. A method comprising:
tuning an activity of a catalyst by incorporating beryllium (Be) into the catalyst, comprising:
adding a first quantity of Be to the catalyst to form a first catalyst material, wherein a 35% conversion temperature of the first catalyst material is lower than a 35% conversion temperature of the catalyst; or
adding a second quantity of Be to the catalyst to form a second catalyst material, wherein a 35% conversion temperature of the second catalyst material is higher than the a 35% conversion temperature of the catalyst.

30. The method of claim 29, wherein the catalyst comprises molybdenum (Mo).

31. The method of claim 30, wherein adding a first quantity of Be comprises adding Be in a molar ratio of Mo:Be of between 1:1 and 1:8; and
adding a second quantity of Be comprises adding Be in a molar ratio of Mo:Be of less than 1.8.

32. (canceled)
